(19) United States
(12) Patent Application Publication
(10) Pub. No.: US 2019/0224322 A1
(43) Pub. Date: Jul. 25, 2019

(54) COMPOSITIONS FOR SMALL MOLECULE THERAPEUTIC AGENT COMPOUNDS

(71) Applicant: Delpor, Inc., South San Francisco, CA (US)

(72) Inventor: Gregory A. WATKINS, Oakland, CA (US)

(21) Appl. No.: 16/336,088

(22) PCT Filed: Sep. 22, 2017
(86) PCT No.: PCT/US2017/053074

§ 371 Date: Mar. 22, 2019

Related U.S. Application Data
(60) Provisional application No. 62/399,083, filed on Sep. 23, 2016.

(51) Int. Cl. A61K 47/16; A61K 47/12; A61K 47/22; A61K 9/00; A61K 31/496; A61K 31/551; A61K 31/407; A61K 31/519

(52) U.S. Cl. CPC A61K 47/16; A61K 47/12; A61K 47/22; A61K 9/0024; A61P 25/18; A61K 31/551; A61K 31/407; A61K 31/519; A61K 31/496

(57) ABSTRACT

A composition comprising an aqueous suspension comprising a small molecule therapeutic agent and an organic acid is described. The small molecule therapeutic agent is a base and has a water solubility at room temperature of less than about 1.0 g/L. The organic acid has a water solubility at room temperature of between 0.1 and 10, has a molar mass of less than 500 grams per mole, and/or maintains a pH of the suspension in its environment of use of between 3.0-6.5. The organic acid enhances the solubility of the small molecule therapeutic agent and when present in stoichiometric excess, the organic acid drives release of the small molecule therapeutic agent into a buffered environment of use for prolonged periods of, for example, six months to one year. Devices comprising the compositions and methods of treatment are also described.

### CROSS-REFERENCE TO RELATED APPLICATIONS

This application claims the benefit of U.S. Provisional Application No. 62/399,083 filed Sep. 23, 2016, incorporated by reference herein.

### TECHNICAL FIELD

The subject matter described herein relates to compositions and formulations for a small molecule therapeutic agent, and to drug delivery devices comprising the compositions and formulations for controlled, sustained delivery of the small molecule therapeutic agent.

### BACKGROUND

Important classes of small molecule drugs exhibit poor water solubility at neutral pH. Although this property may favor oral absorption and tissue penetration, it complicates the development of injectable or implantable sustained delivery systems which rely on passive diffusion as the primary drug release mechanism; low solubility fails to create a concentration gradient sufficient to drive adequate efflux from a reservoir containing an aqueous suspension of the drug, for example. Many insoluble drugs are weak organic bases (i.e., molecules that include at least one functional group such as a primary, secondary, or tertiary amine, aniline, or amidine), and their water solubility improves upon protonation; i.e., when they are converted into a salt. However, such salts are unstable and susceptible to hydrolysis at pH values approaching or exceeding the pKa of the protonated drug. This process complicates diffusion-mediated drug delivery through an implant or depot, since efflux of drug from the formulation must be coupled to a constant influx of buffering species from physiological fluids. Compositions and devices that address these, and other, complications related to sustained and controlled delivery of small molecule therapeutic agents that are weak organic bases, are needed.

### BRIEF SUMMARY

The following aspects and embodiments thereof described and illustrated below are meant to be exemplary and illustrative, not limiting in scope.

In one aspect, a composition, comprising an aqueous suspension is provided. The aqueous suspension comprises a small molecule therapeutic agent that (i) has a water solubility at room temperature of less than about 1 g/L and (ii) is a weak base (i.e., possessing a conjugate acid with a pKa between 6 and 9), combined with a stoichiometric excess of an organic acid that (i) has a water solubility at room temperature of less than about 20 g/L and (ii) maintains a pH of the suspension in its environment of use of between 3-6.5 for a period of at least about 30 days.

In another aspect, a composition, comprising an aqueous suspension is provided. The aqueous suspension comprises a small molecule therapeutic agent that (i) has a water solubility at room temperature of less than about 1 g/L and (ii) is a weak base (i.e., possessing a conjugate acid with a pKa between 5 and 9), combined with a stoichiometric excess of an organic acid that (i) has a water solubility at room temperature between 0.1 and 10 g/L; (ii) has a molecular weight less than 500 grams per mole; and (iii) maintains a pH of the suspension in its environment of use of between 3-6.5 for a period of at least about 30 days.

In another aspect, a composition, comprising an aqueous suspension is provided. The aqueous suspension comprises a small molecule therapeutic agent that (i) has a water solubility at room temperature of less than about 1 g/L and (ii) becomes more soluble upon protonation, combined with a stoichiometric excess of an organic acid that (i) has a water solubility at room temperature of less than about 20 g/L and (ii) maintains a pH of the suspension in its environment of use that is equal to or below the pKa of the protonated drug for a period of at least about 30 days.

In another aspect, a composition, comprising an aqueous suspension is provided. The aqueous suspension comprises a small molecule therapeutic agent that (i) has a water solubility at room temperature of less than about 1 g/L and (ii) becomes more soluble upon protonation, combined with a stoichiometric excess of an organic acid that (i) has a water solubility between 0.1 and 10 g/L; (ii) has a molecular weight less than 500 grams per mole; and (iii) maintains a pH of the suspension in its environment of use that is equal to or below the pKa of the protonated drug for a period of at least about 30 days.

In one embodiment, the aqueous suspension is a heterogeneous mixture comprising the small molecule therapeutic agent and the organic acid, where the organic acid sufficiently dissolves to maintain the pH of the heterogeneous solution in its environment of use at a value equal to or less than physiological pH (˜7.4) for the stated period. In one embodiment, the environment of use is in vivo. In another embodiment, the environment of use is in vitro in a release medium maintained at 37° C.

In one embodiment, the organic acid is present in an amount approximately equal to or above its saturation concentration at the end of the period.

In another embodiment, the organic acid is present in a stoichiometric (molar) amount ranging from about 105% to 1000% relative to the therapeutic agent, but as much as 10,000%. In other embodiment, the organic acid on a molar basis is 110%, 125%, 150%, 175% 200%, 250%, 300%, 350%, 400%, 450%, 500% more than the molar amount of therapeutic agent in the composition.

In another embodiment, the organic acid is crystalline and has a melting temperature of more than about 37° C.

In yet another embodiment, the small molecule therapeutic agent is an antipsychotic medication.

In other embodiments, the antipsychotic medication is risperidone, olanzapine, paliperidone, aripiprazole, brexpiprazole, or asenapine.

In one embodiment, the aqueous suspension comprises, or is manufactured with, an organic acid suspended into a water-based solution, such as an aqueous buffered solution.

In another embodiment, the aqueous suspension comprises, or is manufactured with, a pre-made salt formed between the therapeutic agent and the organic acid, where the acid is present in stoichiometric (molar) excess.

In another embodiment, the therapeutic agent and a stoichiometric (molar) excess of the organic acid are intimately mixed by dissolution into a polar organic solvent such as methanol, ethanol, 1-propanol, 2-propanol, tert-butanol, acetone, 2-butanone, or ethyl acetate, followed by concentration of the intermediate solution to dryness.

In one embodiment, the organic acid is an aromatic carboxylic acid. Exemplary acids, in one embodiment, are those having a carboxylic acid group bound to an unsubstituted benzene or pyridine ring. In one embodiment, the carboxylic acid is selected from the group consisting of benzoic acid, picolinic acid, nicotinic acid, and isonicotinic acid.

In another embodiment, the carboxylic acid is one having a benzene ring and one electron-donating group. In another embodiment, the carboxylic acid has antioxidant properties.

In still another embodiment, the carboxylic acid is selected from the group consisting of o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In another embodiment, the carboxylic acid is one having one benzene ring and two electron donating groups. In another embodiment, the carboxylic acid has antioxidant properties. In one embodiment, and by way of example, the carboxylic acid is vanillic acid.

In yet another embodiment, the carboxylic acid is one having at least two carboxylic acid groups bonded to a benzene ring. In one embodiment, and by way of example, the carboxylic acid is phthalic acid.

In yet another embodiment, the carboxylic acid is one having a carboxylic acid group bonded to a naphthalene or quinoline ring. In one embodiment, and by way of example, the carboxylic acid is selected from the group consisting of 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid.

In another embodiment, the carboxylic acid contains an aromatic ring bearing an electron-donating group selected from the group consisting of hydroxy, methoxy, amino, alkylamino, dialkylamino, and alkyl. In one embodiment, and by way of example, the carboxylic acid is selected from the group consisting of 6-hydroxy-2-naphthoic acid, 6-hydroxy-3-naphthoic acid, 8-hydroxy-2-quinolinecarboxylic acid and 8-hydroxy-7-quinolinecarboxylic acid.

In yet another embodiment, the carboxylic acid is one having one or two carboxylic acid groups directly bonded to a biphenyl ring system. In one embodiment, and by way of example, the carboxylic acid is selected from the group consisting of 2-phenylbenzoic acid, 3-phenylbenzoic acid, 4-phenylbenzoic acid and diphenic acid.

In yet another embodiment, the carboxylic acid is one having one additional electron donating substituent in addition to a hydroxyl group on the carboxylic acid moiety. In one embodiment, and by way of example, the carboxylic acid is selected from the group consisting of 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In still another embodiment, the carboxylic acid is one having a carboxylic acid functional group separated from a benzene, pyridine, naphthalene, or quinoline ring by a chain of 1-4 saturated carbon atoms. In one embodiment, and by way of example, the carboxylic acid is phenylacetic acid or 3-phenylpropionic acid.

In another embodiment, the carboxylic acid is an aliphatic dicarboxylic acid with a 4-8 carbon chain separating the carboxylic acid groups. In one embodiment, and by way of example, the carboxylic acid is selected from the group consisting of adipic acid ((CF₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another embodiment, the carboxylic acid is an unsaturated or polyunsaturated dicarboxylic acid containing 4-10 carbons. In one embodiment, and by way of example, the carboxylic acid is selected from the group consisting of fumaric acid, trans, trans-muconic acid, cis, trans-muconic acid, and cis, cis-muconic acid.

In other embodiments, the carboxylic acid is a cis-cinnamic acid or a trans-cinnamic acid. In still other embodiments, the carboxylic acid is a trans-cinnamic acid with one or two electron-donating groups selected from hydroxy, methoxy, amino, alkylamino, dialkylamino, or alkyl groups. In yet other embodiments, the trans-cinnamic acid is selected from the group consisting of o-coumaric acid, m-coumaric acid, p-coumaric acid, o-methylcinnamic acid, m-methylcinnamic acid, p-methylcinnamic acid, o-methoxycinnamic acid, m-methoxycinnamic acid, p-methoxycinnamic acid, and ferulic acid.

In one embodiment, the organic acid is a phenol or a naphthol substituted with between about 2-5 electron-withdrawing groups selected from F, Cl, Br, I, CN, and NO₂. In one embodiment, and by way of example, the organic acid is pentafluorophenol or 2,4-dinitrophenol.

In another embodiment, the organic acid is a 1,3-dicarbonyl compound containing an acidic CH bond (pKa<8). In one embodiment, and by way of example, the organic acid is 2,2-dimethyl-1,3-dioxane-4,6-dione (Meldrum's acid), cyanuric acid, or barbituric acid.

In still another embodiment, the organic acid is an imide. In one embodiment, and by way of example, the imide is phthalimide or a substituted phthalimide. In another embodiment, the substituted phthalimide has at least one electron-withdrawing substituent.

In yet another embodiment, the organic acid is a hydroxamic acid. In one embodiment, and by way of example, the hydroxamic acid is an aromatic hydroxamic acid containing one hydroxamic functional group bonded directly to an aromatic ring. In one embodiment, the aromatic ring is selected from the group consisting of a benzene ring, a pyridine ring, a naphthalene ring, a quinolone ring, and a biphenyl ring. In still another embodiment, the hydroxamic acid is benzhydroxamic acid. In yet another embodiment, the hydroxamic acid is one containing a hydroxamic functional group separated from an aromatic ring by a chain of 1-4 sp³-hybridized carbon atoms.

In yet another embodiment, the aromatic ring is selected from the group consisting of a benzene ring, a pyridine ring, a naphthalene ring, a quinoline ring, and a biphenyl ring.

In still another embodiment, the hydroxamic acid is a dihydroxamic acid containing two or more hydroxamic acid functional groups bonded directly to a benzene ring, a pyridine ring, a naphthalene ring, a quinoline ring, or a biphenyl ring system.

In other embodiments, the hydroxamic acid contains an aromatic ring that bears an electron donating substituent selected from hydroxy, methoxy, amino, alkylamino, dialkylamino, and alkyl groups.

In other embodiments, the hydroxamic acid is an aliphatic dihydroxamic acid containing 6-10 carbon atoms.

The hydroxamic acid is, in one embodiment, suberohydroxamic acid.

The hydroxamic acid is, in other embodiments, an unsaturated dihydroxamic acid containing 6-10 carbon atoms.

In another embodiment, the aromatic carboxylic acid is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and phthalic acid.

In yet another embodiment, the hydroxy-derivative of cinnamic acid is m-coumaric acid or p-coumaric acid.

In yet other embodiments, the p-coumaric acid is trans-p-coumaric acid.

In other embodiments, the methoxy derivative of cinnamic acid is p-methoxycinnamic acid or m-methoxycinnamic acid.

In still other embodiments, the amino-derivative of benzoic acid is o-amino-benzoic acid (anthranilic acid) or 4-aminobenzoic acid (para-aminobenzoic acid; PABA).

In another embodiment, the methoxy derivative of benzoic acid is 4-methoxybenzoic acid (p-anisic acid), o-anisic acid or m-anisic acid.

In one embodiment, the composition is in a dry form. In another embodiment, the composition is in dry form and hydrates in situ when in its environment of use.

In another aspect, a device comprising a composition as described herein is provided. The device is configured for subcutaneous implantation into a mammal.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) an organic acid that (a) maintains a pH of the formulation when hydrated in its environment of use of between 3.0-6.5 for the delivery period; (b) is present in stoichiometric (molar) excess, relative to the therapeutic agent, and (c) is present at the end of the delivery period in an amount approximately equal to or above its saturation concentration in the formulation when hydrated.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) an organic acid that (a) maintains a pH of the formulation when hydrated in its environment of use equal to or less than the pKa of the protonated drug for the delivery period; (b) is present in stoichiometric (molar) excess, relative to the therapeutic agent, and (c) is present at the end of the delivery period in an amount approximately equal to or greater than its saturation concentration in the formulation when hydrated.

In one embodiment, the formulation is in dry form. In various embodiments, and by way of example, the formulation is a powder, a tablet or a film; or a mixture of two or more powders, tablets, or films.

In another embodiment, the formulation hydrates in the presence of an aqueous solution to form an aqueous suspension. In one embodiment, the aqueous solution is in vivo fluid.

In another embodiment, the small molecule therapeutic agent is released from the device at a rate that provides a therapeutic effect for the period.

In still another embodiment, the organic acid has a water solubility at room temperature of less than about 20 g/L. In still another embodiment, the organic acid has a water solubility at room temperature between 0.1 and 10 g/L and a molar mass less than 500 grams per mole.

In another embodiment, the organic acid has a water solubility at room temperature of less than about 20 g/L and a pKa between 3 and 6. In another embodiment, the organic acid has a water solubility at room temperature between 0.1 and 10 g/L, a molar mass less than 500 grams per mole, and a pKa between 3 and 6.

In another embodiment, two or more organic acids, each with a water solubility of 0.1 to 10 g/L, a molar mass less than 500 grams per mole, and a pKa between 3 and 6 are used in combination.

In yet another embodiment, the organic acid has a melting temperature of greater than about 37° C.

In another aspect, a method for sustained, controlled delivery of a small molecule therapeutic is provided. The method comprises providing a composition or a device as described herein. In some embodiments, the method further comprises administering the device, such as by subcutaneous implantation.

In another aspect, a method for sustained, controlled delivery of an antipsychotic drug is provided, where the method comprises providing a composition or a device as described herein. In some embodiments, the method further comprises administering the device, such as by subcutaneous implantation.

In another aspect, a method to provide maintenance therapy to treat schizophrenia or bipolar disorder is provided, where the method comprises providing a composition or a device as described herein. In some embodiments, the method further comprises administering the device, such as by subcutaneous implantation.

In addition to the exemplary aspects and embodiments described above, further aspects and embodiments will become apparent by reference to the drawings and by study of the following descriptions.

Additional embodiments of the present methods, devices and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are illustrations of a drug delivery device, in assembled form (FIG. 1A) and in unassembled form (FIG. 1B).

FIGS. 1C-1F illustrate a portion of a first exemplary drug delivery device, showing the end cap subassembly in cross sectional in assembled form (FIG. 1C) and in an exploded view (FIG. 1D), and in isometric view when assembled (FIG. 1E). FIG. 1F shows an exploded view of the cap subassembly alone. The numbered elements of the subassembly are 1=cap, 2=porous membrane, 3=seal, 4=retention ring, and 5=drug device reservoir.

FIGS. 1G-1K illustrate a portion of a second exemplary drug delivery device, showing the end cap subassembly in cross sectional in assembled form (FIG. 1G) and in an exploded view (FIG. 1H), and in isometric view when assembled (FIG. 1I). FIGS. 1J-1K show an assembled and exploded view of the cap subassembly alone. The numbered elements of the subassembly are 1=cap, 2=porous membrane, 3=seal, 4=drug delivery device reservoir, and 5=retention ring.

FIG. 2 shows the cumulative release of risperidone, in mg, as a function of time, in days, from drug delivery devices comprising a heterogeneous aqueous formulation comprised of risperidone and 4-aminobenzoic acid (PABA) at risperidone/PABA molar ratios of 1:1 (diamonds); 1:1.5 (squares); 1:2 (closed circles); and 1:2 with membrane surface area reduced by 50% (open circles).

FIG. 3A shows the cumulative release of olanzapine, in mg, as a function of time, in days, from drug delivery devices containing in the device reservoir a heterogeneous aqueous formulation comprised of olanzapine and 4-aminobenzoic acid (PABA, squares) or p-toluic acid (diamonds) at a molar ratio of olanzapine/organic acid 1:1.5, or with no acid as a control (circles).

FIG. 3B shows the cumulative release of olanzapine, in mg, as a function of time, in days, from drug delivery devices containing in the device reservoir a heterogeneous aqueous formulation comprised of olanzapine and 4-aminobenzoic acid (PABA, *) or p-toluic acid (triangles) at a molar ratio of olanzapine/organic acid 2:1, or with no acid as a control (squares).

FIG. 4 shows the plasma concentration of risperidone, in ng/mL, as a function of time, in days, from subcutaneously implanted drug delivery devices comprising in the device reservoir an aqueous formulation of risperidone and 4-aminobenzoic acid (PABA, circles) or sebacic acid (diamonds).

FIG. 5 is a graph showing the cumulative in vitro release (expressed as the percent of total risperidone released into a receiving medium) for various risperidone salts (PABA, squares; terephthalic, diamonds; sebacic, open diamonds; vanillate, triangles; hippurate, x symbols; hydroxyphenylpropionate, open circles; urate, solid circles).

FIG. 6 is graph of the percent of risperidone released on day 15 in the study of Example 5 (FIG. 5) as a function of water solubility, in mg/mL, of the organic acid used in the composition, terephathalic acid, uric acid, sebacic acid, vanillic acid, hydroxyphenylpropionic acid, hippuric acid and PABA.

FIG. 7 is a graph of percent of risperidone released on day 15 in the study of Example 5 (FIG. 5) as a function of pH of the organic acid used in the composition, terephathalic acid, uric acid, sebacic acid, vanillic acid, hydroxyphenylpropionic acid, hippuric acid and PABA, the pH at saturation concentration in an aqueous solution.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein in reference to methods of administration of a small molecule which reduces the frequency of, or delays the onset of, symptoms of a medical condition (e.g., schizophrenia, bi-polar disorder) in a subject relative to a subject not receiving the compound or composition. This can include reversing, reducing, or arresting the symptoms, clinical signs, and underlying pathology of a condition in a manner to improve or stabilize a subject's condition (e.g., controlling schizophrenia symptoms).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Formulations to Enhance the Solubility of a Small Molecule Therapeutic Agent

In one aspect, a composition or formulation in which a small molecule therapeutic agent is solubilized through the use of partially soluble organic acids to improve delivery of the therapeutic agent from a device or drug delivery platform for a sustained period of time. In one embodiment, the composition is an aqueous suspension or slurry. In another embodiment, the composition is a heterogeneous or nonuniform mixture or solution. The solution or mixture can be, in some embodiments, an aqueous mixture or an aqueous heterogeneous mixture. In another embodiment, the composition is in dry form (e.g., lyophilized, spray dried, desiccated, etc.). In these various embodiments, the composition comprises a small molecule therapeutic agent that can function as a Bronsted or Lewis base and an organic acid that has one or more of the following: (i) a water solubility at room temperature (e.g., approximately 25° C.) of less than about 20 g/L or of between about 0.1 to 10 g/L; (ii) a molar mass less than 500 grams per mole; (iii) is present in a stoichiometric (molar) excess relative to the therapeutic agent; and (iv) maintains a pH of the suspension (or solution) in its environment of use approximately equal to or less than the pKa of the protonated therapeutic agent for a period of at least about 30 days. The composition may additionally comprise an aqueous fluid, for example water, buffer or a water-solvent mixture. In embodiments where the composition is in dry form, the aqueous fluid hydrates the composition in situ in its environment of use.

As noted above, the formulations described herein provide solubility of the small molecule therapeutic agent in order to permit delivery for a sustained period. In one embodiment, a sustained period of time intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days. In another embodiment, the sustained period of time intends a period of at least about six months, or nine months, or twelve months.

Also as noted above, the formulations described herein enhance the solubility of the small molecule therapeutic in part by maintaining a particular pH range of the formulation in its environment of use for the stated period of time. In one embodiment, the environment of use is in vivo. For example, the formulation may be part of a drug delivery device that is implanted in vivo and several examples of such devices are provided below. In another embodiment, the environment of use is in vitro in a release medium maintained at about 37° C.

The components of the composition, namely the small molecule therapeutic agent and the organic acid, are now described.

### A. Small Molecule Therapeutic Agents

In one embodiment, the compositions comprise a small molecule therapeutic agent that (i) has a water solubility at room temperature of less than 1.0 g/L and (ii) is an organic base. Reference to “small molecule”, in one embodiment, is to a biologically active molecule that has a molecular weight of less than or equal to 2,000 Daltons, and is generally used in the context of a small molecule drug (therapeutic agent) as distinguished from a protein, polypeptide or peptide therapeutic agent. In another embodiment, the small molecule has a molecular weight of less than or equal 1,000 Daltons or less than or equal to 500 Daltons. In other embodiments, the molecular weight of the small molecule is between 10-2000 Daltons, 10-1000 Daltons, 10-500 Daltons, 50-2000 Daltons, 50-1000 Daltons, 50-500 Daltons, 100-2000 Daltons, 100-1000 Daltons, or 100-500 Daltons.

Small molecule therapeutic agents contemplated include, but are not limited to, agents that are weak organic bases (i.e., possessing conjugate acids with pKas between 6 and 9 or between 5 and 9) and a potency such that a 30-60 day dose can be contained in a delivery device implanted into a human.

By way of example, therapeutic agents that include a primary, secondary, or tertiary amine, an aniline or aniline derivative, or amidine functional group are contemplated as small molecule therapeutic agents that are organic bases. It will be appreciated that therapeutic agents having a structure containing more than one of these functional groups are contemplated. Examples of aniline derivatives include analogues of aniline where the phenyl group is substituted with, for example, a methyl group (toluidine), a halogen such as chlorine (2-chloroaniline, 3-chloroaniline, 4-chloroaniline), an amino group (4-aminobenzoic acid, or 2-aminobenzoic acid, or 3-aminobenzoic acid), a nitro group (e.g., 2-, 3-, or 4-nitroaniline), and many others.

In one embodiment, the small molecule therapeutic agent is an antipsychotic drug, including atypical antipsychotics. In another embodiment, the small molecule therapeutic agent has activity to treat a disease of the central nervous system. Exemplary agents include, but are not limited to, risperidone, olanzapine, asenapine, aripiprazole, or brexpiprazole.

In one embodiment, small molecule drug i) is poorly water soluble at physiological pH (˜7.4) and ii) functions as a Bronsted or Lewis base. As will be described below, in the presence of an aqueous fluid and a stoichometric excess of an organic acid that i) has a solubility in water between 0.1 and 10 g/L or less than or equal to 20 g/L at 25° C., and ii) dissolves at least partially in the presence of the drug and a physiological buffer, a suspension or slurry is produced with a pH (within the aqueous fraction) approximately equal to or less than the pKa of the protonated drug.

### B. Organic Acids

The composition, in addition to a small molecule therapeutic agent, comprises an organic acid or combination of organic acids. The organic acid is one that has one or more of the following features: (i) a water solubility at room temperature of between 0.1 and 10 g/L or of less than about 20 g/L; (ii) a molar mass less than 500 grams per mole; (iii) is present in stoichiometric excess relative to the therapeutic agent; and (iv) maintains a pH of the suspension or solution in its environment of use approximately equal to or less than the pKa of the protonated small molecule therapeutic agent for a period of at least about 30 days. As described above, the compositions enhance the solubility of the small molecule therapeutic agent, permitting use of the composition in a drug delivery platform that provides sustained release for an extended period of time. Excess acid (on a stoichiometric basis, relative to the therapeutic agent) intercepts physiological buffering species that would otherwise drive hydrolysis of the pharmacologically active salt. Examples of organic acids for use in the compositions are now described.

In a first embodiment, the organic acid is a carboxylic acid. Examples include aromatic carboxylic acids where a carboxylic acid group is bonded directly to an aromatic ring. For example, the aromatic carboxylic acid can have one carboxylic acid group bound to an unsubstituted benzene or pyridine ring. Examples include benzoic acid, picolinic acid, nicotinic acid, or isonicotinic acid. In another example, the aromatic carboxylic acid is one having a benzene ring and one electron-donating group with antioxidant properties. Specific examples include o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In yet another example, the aromatic carboxylic acid is one having a single benzene ring and two electron donating groups with antioxidant properties. A specific example is vanillic acid. In still another example, the aromatic carboxylic acid is one having two or more carboxylic acid groups bonded to a benzene ring. A specific example is phthalic acid.

In another example, the aromatic carboxylic acid is one having one carboxylic acid group bonded to a naphthalene or quinoline ring. Examples include 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid. A further grouping of acids of this type, with one carboxylic acid group bonded to a naphthalene or quinoline ring, include those containing an additional electron-donating group, such as a hydroxy, methoxy, amino, alkylamino, dialkylamino, or alkyl group. Examples of acids in this grouping include 6-hydroxy-2-naphthoic acid, 6-hydroxy-3-naphthoic acid, 8-hydroxy-2-quinolinecarboxylic acid, 8-hydroxy-7-quinolinecarboxylic acid, and isomers of each.

In another exemplary embodiment, the carboxylic acid is one having one carboxylic acid group bonded to a naphthalene or quinoline ring and an electron donating substituent in addition to hydroxyl group on the carboxylic acid moiety. Examples include 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In another exemplary embodiment, the acid is a di- or tri-carboxylic acid having two or three carboxylic acid groups bonded to a naphthalene or quinoline ring. Examples include 1,4-naphthalenedicarboxylic acid and 2,6-naphthalenedicarboxylic acid.

In another exemplary embodiment, the carboxylic acid is one having one or two carboxylic acid groups directly bonded to a biphenyl ring system. Examples include 2-phenylbenzoic acid, 3-phenylbenzoic acid, 4-phenylbenzoic acid and diphenic acid.

In another exemplary embodiment, the carboxylic acid is one having a carboxylic acid functional group separated from a benzene, pyridine, naphthalene, or quinoline ring by a chain of 1-4 saturated carbon atoms. Examples of acids in this embodiment include phenylacetic acid and 3-phenylpropionic acid.

In another exemplary embodiment, the carboxylic acid is an aliphatic dicarboxylic acid with 6-10 carbon atoms, such as adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another exemplary embodiment, the carboxylic acid is an unsaturated or polyunsaturated dicarboxylic acid containing 4-10 carbons. Examples of acids in this embodiment include fumaric acid, trans, trans-muconic acid, cis, trans-muconic acid, and cis, cis-muconic acid.

In another exemplary embodiment, the carboxylic acid is a cis- or trans-cinnamic acid. In one embodiment, the trans-cinnamic acid has one or two electron-donating groups selected from hydroxy, methoxy, amino, alkylamino, dialkylamino, or alkyl groups. Examples include o-coumaric acid, m-coumaric acid, p-coumaric acid, o-methylcinnamic acid, m-methylcinnamic acid, p-methylcinnamic acid, o-methoxycinnamic acid, m-methoxycinnamic acid, and p-methoxycinnamic acid, and ferulic acid.

In another embodiment, the organic acid is a phenol or a naphthol substituted with between about 2-5 electron-withdrawing groups selected from —F, —Cl, —Br, —I, —CN, —CHO (aldehyde), —COR (ketone), and NO₂. Examples include 2,4-dinitrophenol.

In another embodiment, the organic acid is a 1,3-dicarbonyl compound containing an acidic CH bond (pKa<8). Examples include 2,2-dimethyl-1,3-dioxane-4,6-dione (Meldrum's acid), cyanuric acid, or barbituric acid.

In another embodiment, the organic acid is an imide, such as phthalimide. In one embodiment, the phthalimide is substituted with at least one electron-withdrawing substituent.

In another embodiment, the organic acid is a hydroxamic acid. The hydroxamic acid may be, in some embodiments, an aromatic hydroxamic acid containing one hydroxamic functional group bonded directly to an aromatic ring. The aromatic ring is selected from the group consisting of a benzene ring, a pyridine ring, a naphthalene ring, a quinoline ring, and a biphenyl ring. Examples include benzhydroxamic acid. The hydroxamic acid can also be one containing a hydroxamic functional group separated from an aromatic ring by a chain of 1-4 sp³-hybridized carbon atoms. Dihydroxamic acids containing two or more hydroxamic acid functional groups bonded directly to a benzene, pyridine, naphthalene, quinoline, or biphenyl ring system are also contemplated. In addition, substituted derivatives of the hydroxamic acids described above that contain electron donating substituents such as hydroxy, methoxy, amino, alkylamino, dialkylamino, or alkyl groups are contemplated. Also contemplated are aliphatic dihydroxamic acids containing 6-10 carbon atoms, such as suberohydroxamic acid, and unsaturated dihydroxamic acids containing 6-10 carbon atoms.

The organic acids for use in the compositions described herein are preferably those with a water solubility at room temperature between 0.1 and 10 g/L or, alternatively, of less than about 20 g/L. In another embodiment, the organic acids for use in the compositions described herein have a molar mass less than 500 grams per mole. In another embodiment, the organic acids for use in the compositions described herein are non-polymeric or non-oligomeric. In another embodiment, the organic acids for use in the compositions described herein do not have a polymeric or oligomeric backbone and/or are not attached to a polymeric or oligomeric backbone. In another embodiment, the acid has a water solubility at room temperature of less than about 20 g/L and a pKa value between about 3 and 6, more preferably a pKa value of between about 3-5.5 or between about 3.5-5.5. In other embodiments, the organic acid is crystalline and has a melting temperature of more than about 37° C.

Compositions comprising a molar excess of an organic acid and a small molecule therapeutic agent are prepared by mixing the organic acid and the therapeutic agent together in a suitable solvent. In some embodiments, the solvent is an aqueous fluid, such as a buffer or a water-organic solvent mixture. In a preferred embodiment, the organic acid is present in an amount such that at the end of the delivery period, it remains at or above its saturation concentration within its environment of use.

Compositions were prepared with the following organic acids listed in Table 1, and pH values were measured.

TABLE 1

| Formulation | In vitro pH | Solubility (g/L) | pKa |
|---|---|---|---|
| Citric | 2.04 | 10 | 3.13 |
| rac-Mandelic | 2.42 | 158.7 | 3.85 |
| R-Mandelic | 2.45 | 158.7 | 3.85 |
| Benzilic | 3.02 | 2 | 3.05 |
| Nicotinic | 3.68 | 18 | 4.75 |
| m-Coumaric | 3.95 | 1.04 | 4.01 |
| PABA | 4.21 | 5.9 | 4.65 |
| trans-Cinnamic | 4.35 | 0.5 | 4.44 |
| p-Coumaric | 4.36 | 1-10 | 4.64 |
| m-Methoxycinnamic | 4.49 | 4.46 | 4.47 |
| 4-Chlorobenzoic | 4.81 | 0.077 | 3.98 |
| p-Anisic | 5.05 | 0.4 | 4.34 |
| p-Methoxycinnamic | 5.37 | 0.712 | 4.04 |
| Cholic | 5.65 | 0.05 | 5.07 |
| 4-Methylcinnamic | 6.13 | | |
| 4-Chlorocinnamic | 6.34 | | 4.41 |
| Sebacic | 6.61 | 0.25 | 4.72 |
| Control | 7.40 | | |

In embodiments where the composition is within a reservoir of a drug delivery device, it will be appreciated that the device when placed in its environment of use is open to the environment of use. That is, the environment of use and the composition in the device are in fluid communication via the pore or porous membrane in the drug delivery device. The compositions described herein include the organic acid in the form of a suspension or slurry, given its limited water solubility. The organic acid is present in the composition in an amount above its saturation concentration, and in accord with another embodiment, the organic acid is present in the composition at the end of the delivery period in an amount at or above its saturation concentration. In this way, the composition maintains the desired pH of the suspension or heterogeneous solution of between 3.0-6.5, preferably 2.75-5.75, more preferably 2.8-5.6, preferably 2.9-5.6, preferably 3.1-5.5, 3.2-5.5, 3.3-5.5, 3.4-5.5, 3.5-5.5, 3.1-5.4, 3.2-5.4, 3.3-5.4, 3.4-5.4, 3.5-5.4, 3.1-5.3, 3.2-5.3, 3.3-5.3, 3.4-5.3, 3.5-5.3, 3.1-5.2, 3.2-5.2, 3.3-5.2, 3.4-5.2, 3.5-5.2, 3.1-5.1, 3.2-5.1, 3.3-5.1, 3.4-5.1, 3.5-5.1, 3.1-5.0, 3.2-5.0, 3.3-5.0, 3.4-5.0, 3.5-5.0, 3.5-5.5 or 3.5-6.0.

In another embodiment, the organic acid is crystalline and has a melting temperature of more than about 37° C. Such organic acids remain in solid form in an in vivo environment of use to provide a heterogeneous mixture or suspension of the organic acid in the composition for the period of delivery time.

In another embodiment, the molar excess of the organic acid ranges from 101%-900%, 101%-800%, 101%-700%, 101%-600%, 101%-500%, 101%-400%, 101%-300%, 101%-200%, 150%-1000%, 150%-900%, 150%-800%, 150%-700%, 150%-600%, 150%-500%, 150%-400%, 150%-300%, 150%-200%. 200%-1000%, 200%-90° %, 200%-800%, 200%-700%, 200%-600%, 200%-500%, 200%-400%, 200%-300%, 150%-10000%, or 200%-10000%.

### Delivery Device

In another aspect, a drug delivery device for administration of a composition or aqueous suspension as described herein is provided. The drug delivery device can be any implantable device, based on, for example, diffusive, erodible or convective systems, e.g., diffusional systems, osmotic pumps, electro-diffusion systems, electro-osmosis systems, electromechanical systems, and the like. In one embodiment, a controlled drug delivery device is utilized, for controlled, extended delivery of the composition for a period of time. The term “controlled drug delivery device” is meant to encompass any device wherein the release (e.g., rate, timing of release, dosing period) of drug or other desired substance contained therein is controlled by or determined by the device itself (wholly or in part) and not solely the environment of use Several non-limiting examples are described.

In one embodiment, the drug delivery device is one having a housing member that defines a reservoir in which the compositions and/or the aqueous suspensions described above are retained. The housing member is of a size and shape that is suitable for implantation into the body. A cylindrical shape is preferable for subcutaneous implantation using a cannula or trocar. The outer diameter of a cylindrically shaped housing member would preferably be in the range of 2 mm to 6 mm and the length in the range of about 10 mm to about 50 mm. The composition or aqueous suspension, in one embodiment, is initially present in a dry form within the reservoir of the device. For example, the aqueous suspension comprising the small molecule therapeutic agent and the organic acid is prepared and subsequently spray dried, milled or lyophilized to provide a dried form of the aqueous suspension. Alternatively, the individual components in dried form—i.e., the therapeutic agent as a dry solid and the organic acid as a dry solid—are mixed in the correct proportions to provide upon later hydration the desired aqueous suspension. Alternatively, the therapeutic agent and the organic acid may be co-dissolved within a suitable organic solvent such as methanol, ethanol, 1-propanol, 2-propanol, tert-butanol, acetone, 2-butanone, or ethyl acetate, followed by concentration to yield a dried powder suitable for resuspension into an aqueous medium. The dried form of the composition can be tableted or pelleted, loaded in the device and hydrated in situ upon subcutaneous implantation of a device containing the dried composition, or the composition can be hydrated at the time of subcutaneous implantation by a clinician introducing a liquid (e.g. a physiological buffer, isotonic saline, phosphate buffered saline, or aqueous propylene glycol) to a reservoir or matrix containing the composition. The liquid can be provided as part of a kit comprising the drug delivery device and a vial comprising a hydration liquid.

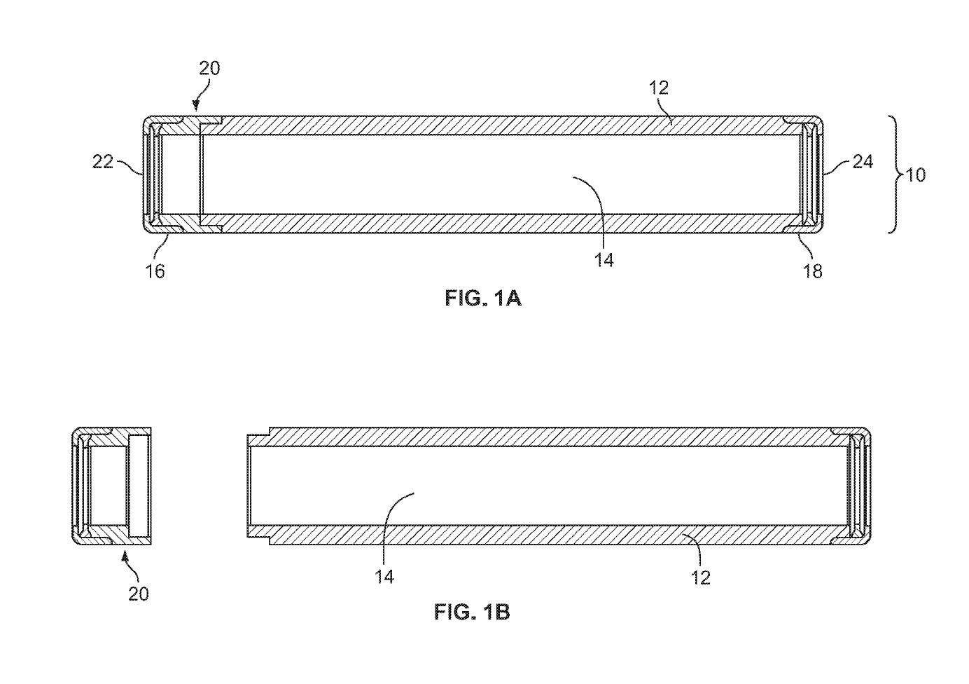

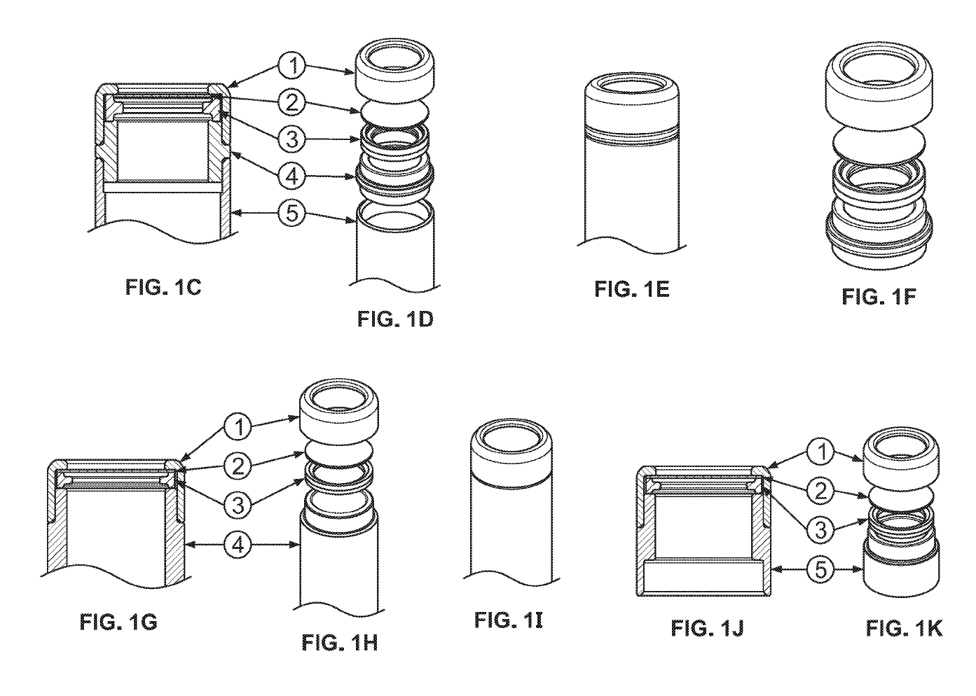

An example of a drug delivery device is provided in FIGS. 1A-1B. FIG. 1A illustrates a device 10, assembled and ready for implantation, in an anatomical compartment of a subject, such as under the skin or in the peritoneal cavity. The device is comprised of a non-erodible housing member 12 that defines an internal compartment or reservoir 14. Contained within the reservoir is a composition or formulation as described herein. Housing member 12 has first and second ends, 16, 18. First end 16 is sealed with a fluid-tight end-cap 20, seen best in FIG. 1B that illustrates device 10 in its unassembled form. End cap 20 may optionally comprise a porous membrane or semi-permeable membrane or porous partition 22. Second end 18 is fitted with a porous membrane, semi-permeable membrane, or porous partition 24.

FIGS. 1C-1K illustrate the end caps and end cap subassembly portions of the exemplary drug delivery devices. The numbered elements of the subassembly illustrated in FIGS. 1C-1F are 1=cap, 2=porous membrane, 3=seal, 4=retention ring, and 5=drug device reservoir. The numbered elements of the subassembly illustrated in FIGS. 1G-1K are 1=cap, 2=porous membrane, 3=seal, 4=drug delivery device reservoir, and 5=retention ring.

The device interior contains a formulation comprising a small molecule drug that is i) poorly water soluble at physiological pH (˜7.4) and ii) can function as a Bronsted or Lewis base. The drug when combined with a stoichiometric excess of an organic acid that i) has a solubility in water between 0.1 and 10 g/L or of less than or equal to 20 g/L at 25° C., and ii) dissolves at least partially in the presence of the drug and a physiological buffer, produces a suspension or slurry with a pH (within the aqueous fraction) approximately equal to or less than the pKa of the protonated drug.

As used herein, the terms “porous membrane” and “porous partition” intend a structural member that has a plurality of pores in the nanometer or micrometer (μm) range, preferably in the 0.1-100 μm or 0.1-200 μm range. The porous partition permits passage of the therapeutic agent in its soluble form from the formulation contained within the reservoir. The porous partition can also permit passage of the organic acid that is part of the formulation in its soluble form. The porous partition in a preferred embodiment retains the therapeutic agent and/or the organic acid in their insoluble forms. That is, the therapeutic agent and/or the organic acid in insoluble form preferably do not pass through the pores of the porous partition. The drug delivery device is described in detail in U.S. 2011/0106006, which is incorporated by reference herein.

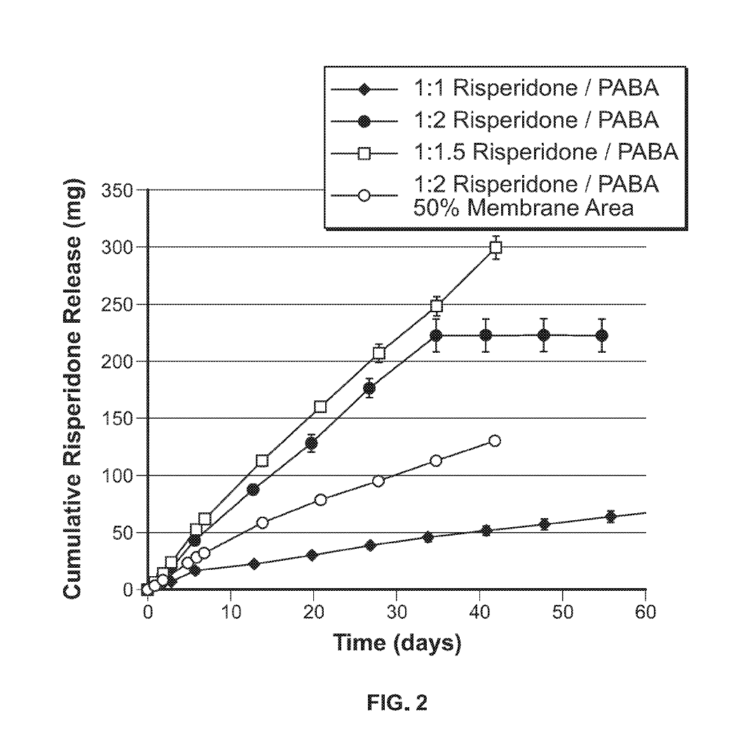

Studies were conducted to evaluate the release rate and kinetic order of release from drug delivery devices containing in the device reservoir compositions comprised of a small molecule therapeutic agent and an organic acid. As described in Examples 1 and 2, compositions of risperidone with various organic acids and of olanzapine with two different organic acids were prepared. Risperidone was selected as a model therapeutic agent due to its potency and insolubility in water as a neutral free base (>10000 volumes of water per volume of drug at 20-25° C.). In the study with risperidone, the drug was compounded with p-aminobenzoic acid (PABA) at acid:drug ratios of 1:1, 1.5:1, or 2:1 (molar basis) to give a stoichiometric excess of organic acid in each formulation. The dry formulations were loaded into the reservoir of delivery devices, hydrated, and incubated within dilute phosphate buffered saline. Release of risperidone was evaluated over a 30 day period and results are shown in FIG. 2.

FIG. 2 shows cumulative release of risperidone, in mg, as a function of time, in days, from drug delivery devices comprising a heterogeneous aqueous formulation comprised of risperidone and 4-aminobenzoic acid (PABA) at risperidone/PABA molar ratios of 1:1 (diamonds); 1:1.5 (squares); 1:2 (closed circles). In one set of devices containing a 1:2 risperidone/PABA formulation, the membrane surface area was reduced by about 50% (open circles). The addition of the organic acid, PABA, to the formulation increased the release rate of therapeutic agent and also provided a more constant rate of release, approaching zero-order kinetics for the delivery period, relative to the control formulations. Devices containing a composition of 1.5:1 or 2:1 PABA/risperidone generated relatively similar output profiles to each other, provided that the membrane surface area of the device was held constant. A reduction in membrane surface area by approximately 50% produced a corresponding reduction in output rate for systems loaded with the 2:1 PABA/risperidone formulation. Note that the device with the 1:2 risperidone/PABA molar ratio plateaus at about day 32 as the device runs out of drug.

In summary, the control formulation (risperidone/PABA salt, no excess acid; diamonds) produced a slow release rate that decreased over time (i.e., non-linear release kinetics) from devices equipped with a maximal membrane surface area. Formulations comprising a 1.5:1 or 2:1 mole ratio of acid to drug (squares, closed circles, respectively) produced a higher rate of drug release relative to the formulation with organic acid not in stoichiometric excess. Devices comprising a 2:1 organic acid/risperidone formulation and approximately half of the membrane surface area produced approximately half of the output rate of devices equipped with 100% of the available surface area and the same formulation.

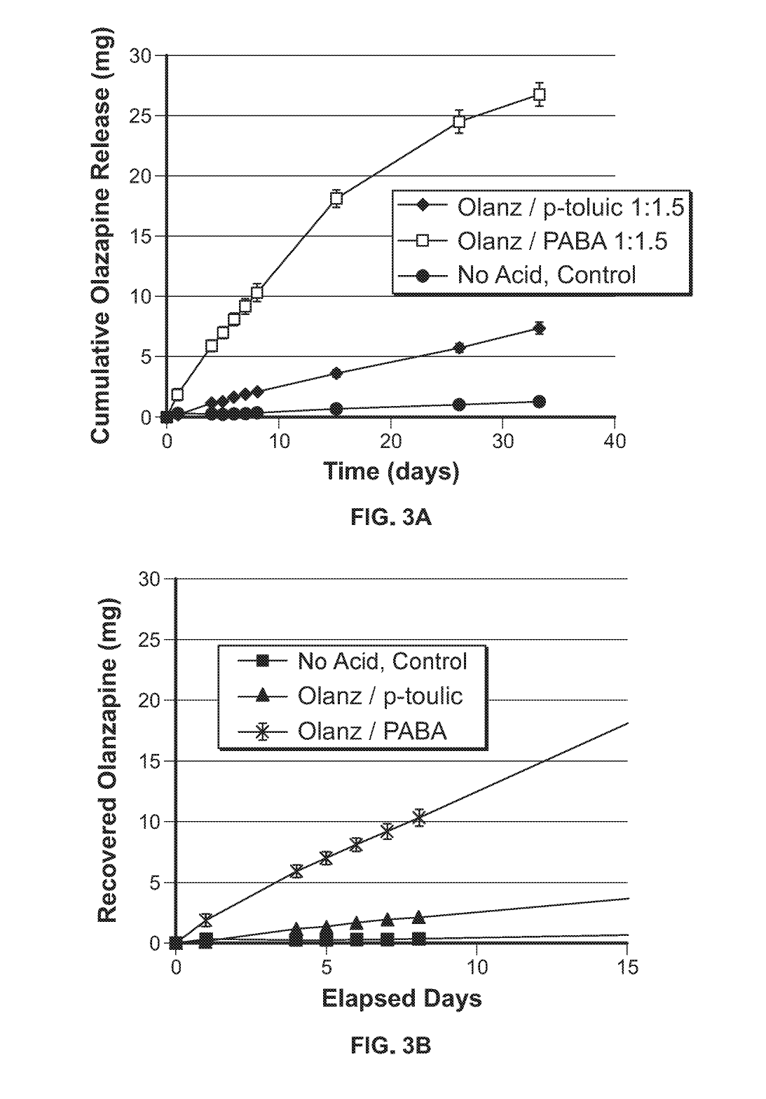

Results for a similar study (Example 2) with olanzapine are shown in FIG. 3A, where the cumulative release of olanzapine, in mg, as a function of time, in days, from drug delivery devices containing in the device reservoir a heterogeneous formulation comprised of olanzapine and 4-aminobenzoic acid (PABA, squares) or p-toluic acid (diamonds) at a molar ratio of olanzapine/organic acid 1:1.5, or with no acid as a control (circles). Olanzapine is a poorly water soluble base. When formulated with a stoichiometric excess (mole ratio of 1.5:1) of an organic acid (PABA or p-toluic acid), an increased release rate and constant rate of release were observed. The different organic acids produced substantially different release rates, likely a reflection of the proximity of the formulation pH values (4.5-5.0) to the reported pKa value of doubly protonated olanzapine (pKa1=5.0; pKa2=7.4).

FIG. 3B shows results for another study like that described in Example 2, expect that the drug delivery devices were filled with a heterogeneous aqueous formulation comprised of olanzapine and 4-aminobenzoic acid (PABA, *) or p-toluic acid (triangles) at a molar ratio of olanzapine/organic acid 2:1. The in vitro cumulative release of olanzapine, in mg, as a function of time, in days, from drug delivery devices is shown in FIG. 3B, where devices comprising olanzapine and PABA (* symbols) released more rapidly than devices comprising a formulation with p-toluic acid (triangles). Devices with no acid—i.e., with olanzapine only, as a control released drug slowly over the 15 day test period (squares).

In summary, little olanzapine free base (<1 mg total) was released from control devices (circles) over the study or treatment period. Devices containing formulations with a 1:1.5 or 1:2 molar ratio of drug to organic acid—PABA (squares) or p-toluic acid (diamonds)—achieved a release rate greater than the control devices, as well as linear release kinetics. In the case of olanzapine, different acid additives produced substantially different release rates; for instance, PABA generated a faster release rate than p-toluic acid. In view of this data, a skilled artisan can appreciate that the release rate can be tailored by selection of the organic acid in the formulation, as well as the molar ratio of drug to organic acid.

In one embodiment, a formulation comprising a small molecule therapeutic agent and an organic acid, with the organic acid present in a stoichiometric amount or in stoichiometric excess, provides an increase in the release rate of the small molecule therapeutic agent of at least 10%, 15%, 20%, 25%, 30%, 35%, 40% or 50% compared to a formulation of the small molecule therapeutic agent with no organic acid or with less than a stoichiometric amount of organic acid. In one embodiment, the increased rate of release is for a period of at least 14 days, at least 2 weeks, at least 30 days or at least 45 days or at least 60 days or at least 90 days or at least 180 days. In another embodiment, the increased rate of release approaches zero-order kinetic release for the period.

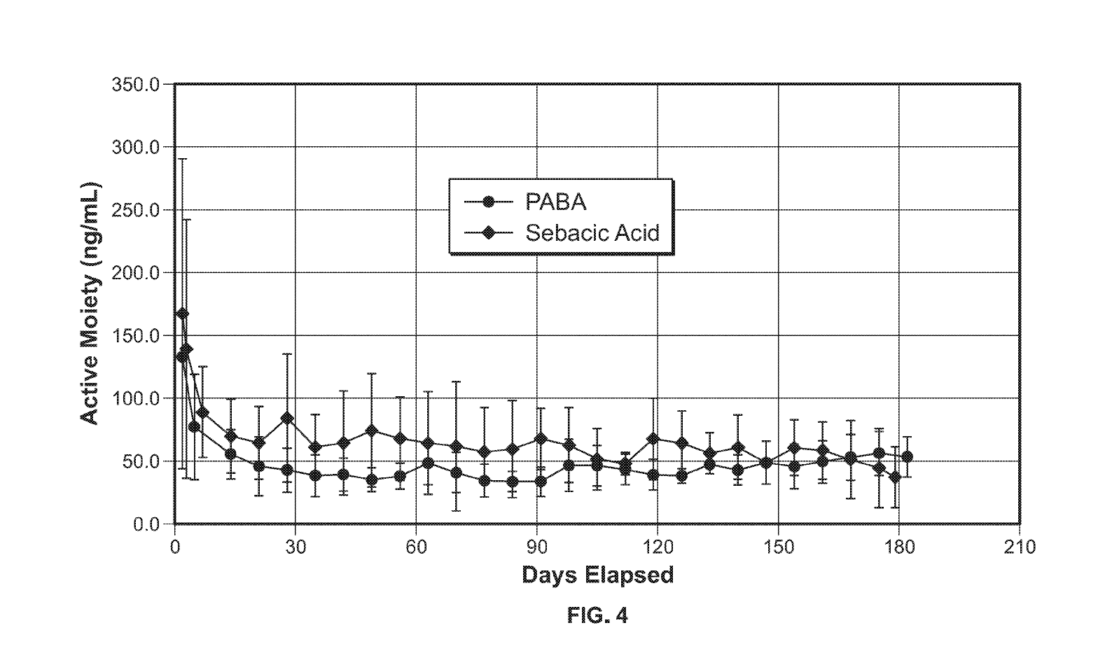

Another study is described in Examples 3-4 where drug delivery devices were manufactured to comprise in the device reservoir a dry tablet of risperidone base and PABA (Example 3) or sebacic acid (Example 4). Tablets comprised of risperidone base and the organic acid in a 1.5:1 weight ratio or a 1:1 weight ratio were prepared by dissolving the drug and organic acid together in a solvent and drying to remove the solvent. The dried drug-organic acid mixture was pulverized and the resulting powder mixed with a binding agent (polyvinylperolidone) and a lubricant (stearic acid) and tablets were pressed. The tablets were loaded into a drug delivery device. Immediately before implantation in vivo each device was filled with sterile phosphate-buffered saline (PBS) to hydrate the tablet. The devices were implanted, and blood samples were obtained for pharmacokinetic (PK) analysis and local safety was assessed for six months. Results are shown in FIG. 4, where the plasma concentration of risperidone, in ng/mL, as a function of time, in days, for the devices with an aqueous formulation of risperidone and 4-aminobenzoic acid (PABA, circles) and for the devices with an aqueous formulation of risperidone and sebacic acid (diamonds). With regard to the devices filled with risperidone and PABA (FIG. 4, circles), plasma levels of risperidone active moiety (risperidone plus its active metabolite 9-OH risperidone) peaked in the first few days and then reached a steady state plasma level of about 50 ng/mL for the six-month implantation period. Mass balance analysis revealed that devices removed after six months released drug at an average rate of 0.70 mg/day and contained an average of 108 mg of unreleased risperidone. These findings indicate that the devices would have operated for another 154 days in vivo for a total operating period of 337 days. To extend the time period of operation the device reservoir can be sized and filled with drug and organic acid sufficient for the period of delivery at a desired rate. For example, to create a 12-month system the reservoir length is increased by 10% from 40.0 mm to 44.0 mm. Accordingly, the dose rate is scaled by increasing the diameter of the device, or by implanting more than one device per subject.

With regard to the devices filled with risperidone and sebacic acid (FIG. 4, diamonds), plasma levels of risperidone active moiety (risperidone plus its active metabolite 9-OH risperidone) peaked in the first few days and then reached a steady state maintaining a plasma level of 50-60 ng/mL for 6 months. Mass balance analysis revealed that devices removed after 6 months released drug at an average rate of 0.80 mg/day and contained an average of 26 mg of unreleased risperidone. These findings indicate that the devices would have operated in vivo for another 32 days for a total operating period of 7 months.

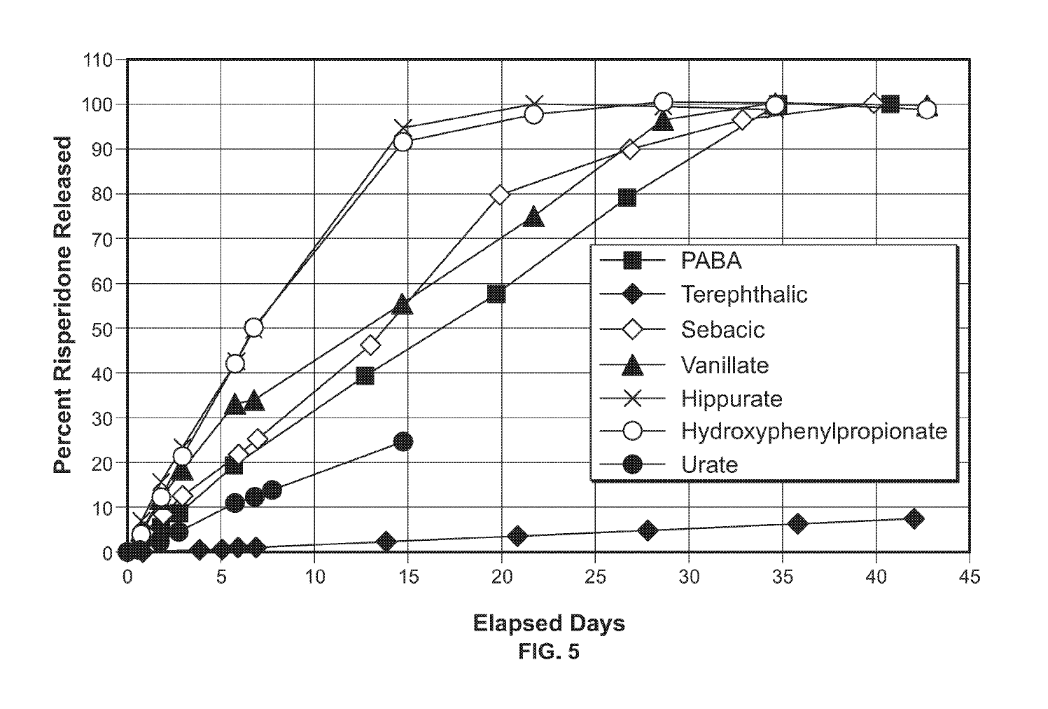

Example 5 describes a study where compositions comprised of various risperidone salts were prepared by dissolving the drug and a two-fold molar excess of a selected organic acid in methanol. The solvent was removed and the dried cake was further dried, pulverized, and in some cases tableted. The dried drug salt was placed into reservoirs of drug delivery devices. The loaded devices were hydrated and placed in 100 mL of PBS at 37° C. Release of risperidone was measured by taking aliquots of the receiving buffer and analyzing for risperidone concentration. FIG. 5 presents the cumulative in vitro release (expressed as the percent of total loaded drug released into a receiving medium) for various risperidone salts (PABA, squares; terephthalic, diamonds; sebacic, open diamonds; vanillate, triangles; hippurate, x symbols; hydroxyphenylpropionate, open circles; urate, solid circles). As can be seen, the slopes of the curves are different indicating different rates of release. Terephthalic (diamonds) and uric acid (solid circles) addition salts produced inadequate output achieving only 2.6% and 16% output in 15 days. The risperidone salts of hippuric acid (x symbols) and hydroxyphenyl propionic acid (open circles) achieved release of risperidone, respectively, of 94% and 92% after 15 days. The risperidone salts of sebacic acid (open diamonds), vanillic acid (triangles) and PABA (squares) produced intermediate rates of risperidone release, with between about 40-60% of the total loaded drug amount released in about 15 days. Accordingly, in one embodiment, the composition of therapeutic agent and organic acid provides release of the therapeutic agent such that at least about 40%, 50%, or 60%, is released in vitro in about 15 days. In another embodiment, the composition of therapeutic agent and organic acid provides release of the therapeutic agent such that no more than about 30% or 40% is released in vitro in about 15 days. In another embodiment, the composition of therapeutic agent and organic acid provides release of the therapeutic agent such that between about 40-50% is released in vitro in about 15 days.

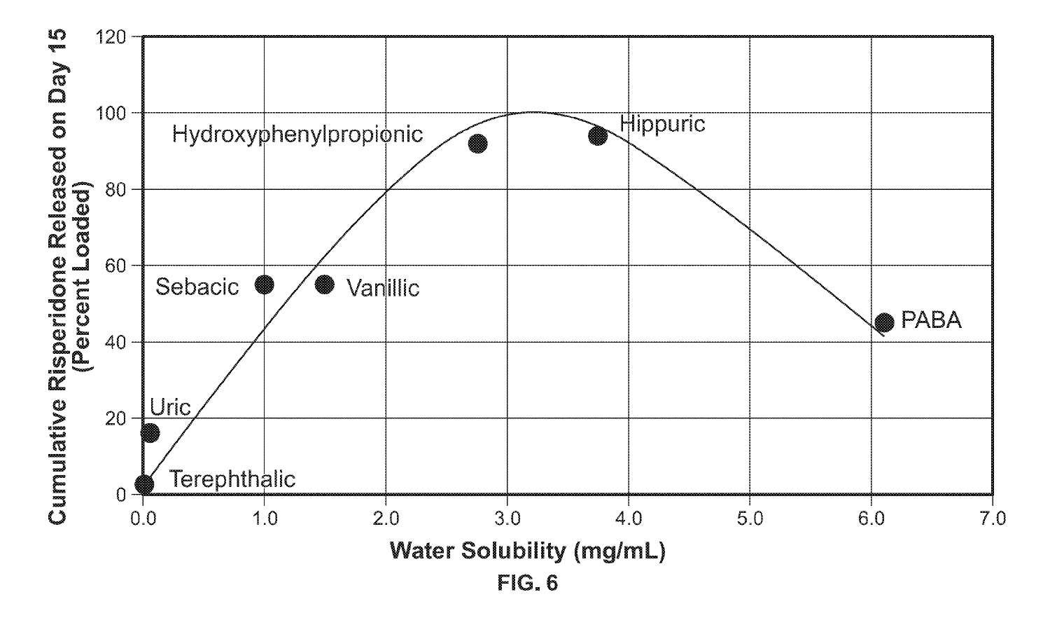

The rates of in vitro release of the risperidone salts described in Example 5 and shown in FIG. 5 are related to the intrinsic water solubility of the acid. The water solubility of the acids used in Example 5 and their respective risperidone release rates into buffer from a device (expressed as the cumulative percent total risperidone released following 15 days incubation at 37° C.) are listed in Table 2. These data are plotted in FIG. 6. The highest risperidone release rate occurs when the drug is combined with an acid with an intrinsic water solubility between about 1.0 to 6.0 mg/mL. The peak release is seen for risperidone salts of hippuric acid and 3-(4-hydroxyphenyl)propionic acid which exhibit water solubilities of between about 2.5 to 4.0 mg/mL at approximately 25° C. This data suggests that acids that have water solubilities less than about 1 g/L do not maintain a sufficiently low pH within the device, whereas acids with water solubilities substantially greater than 6 g/L are released from the device too rapidly, and therefore cannot sustain output of the drug over prolonged periods of time.

TABLE 2

| Acid Addition Salt of | Water Solubility | Percent Risperidone |
|---|---|---|
| Risperidone | (mg/mL) | Released at Day 15* |
| Terephthalic acid | 0.02 | 2.6 |
| Uric acid | 0.06 | 16 |
| Sebacic acid | 1.00 | 55 |
| Vinallic acid | 1.50 | 55 |
| Hydroxyphenylpropionic acid | 2.76 | 92 |
| Hippuric acid | 3.75 | 94 |
| PABA | 6.11 | 45 |

*See Example 5 and FIG. 5

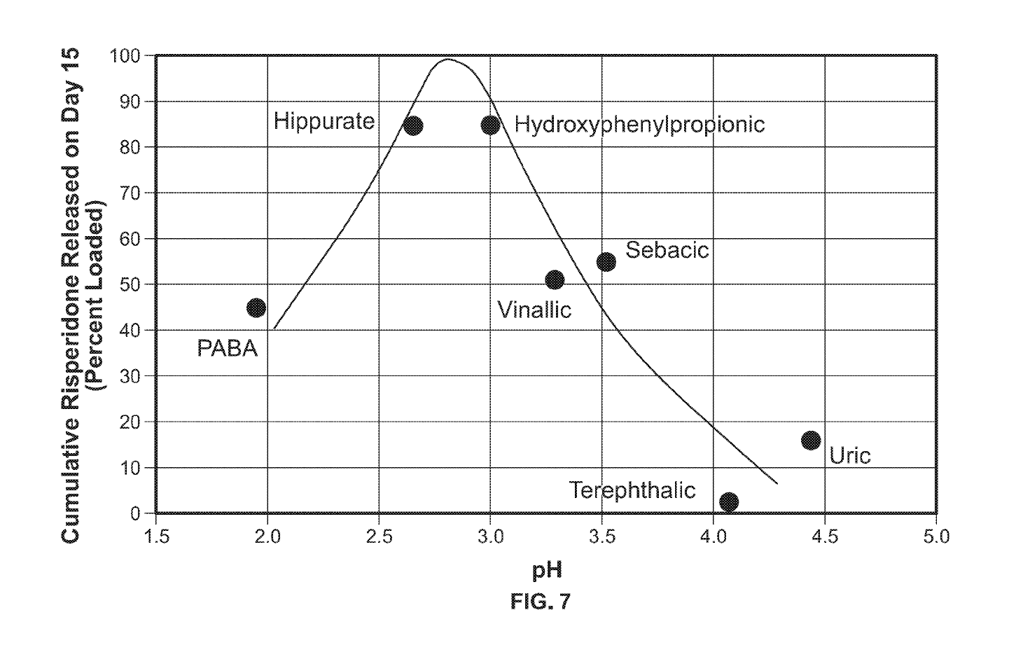

The rates of in vitro release of the risperidone salts listed in Example 5 are also related in part to the pH of a saturated aqueous solution of the acid. The pH at saturating concentrations of the acids used in Example 5 and their respective risperidone release rates (expressed as the cumulative percent total risperidone released following 15 days incubation at 37° C.) are shown in FIG. 7. The highest risperidone release occurs when the drug is combined with an acid which exhibits a pH at a saturating concentration between about 2.0 and 3.7. The peak release is seen for risperidone salts of hippuric acid and 3-(4-hydroxyphenyl)propionic acid which exhibit pH values of 2.6 and 3.0, respectively. Accordingly, in one embodiment, the composition is comprised of a therapeutic agent and an organic acid with a pH at saturation in an aqueous solution of between about 2.0-3.7, or between about 2.1-3.6, between about 2.1-3.5, between about 2.2-3.5 between about 2.2-3.4, between about 2.3-3.4, between about 2.4-3.3, between about 2.5-3.2, between about 2.5-3.1, between about 2.5-3.0, between about 2.6-3.2, between about 2.6-3.1, or between about 2.6-3.0.

Other drug delivery devices are known in the art. The compositions described herein are useful for a variety of devices, including those comprise a drug reservoir for retaining the small molecule therapeutic agent and organic acid formulation and those that have a substrate or matrix that can hold or contain the formulation. Controlled drug release devices suitable for use in the present invention generally can provide for delivery of the drug from the device at a selected or otherwise patterned amount and/or rate to a selected site in the subject. The drug delivery device must be capable of containing an amount of the formulation to provide a therapeutically effective amount of the small molecule for the period of therapy. The period of delivery will vary according to the therapeutic agent, the condition being treated, and the individual patient. In one embodiment, the period of delivery, also referred to herein as a sustained period of time, intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days. In other embodiments, the period of time is from about 2 hours to about 72 hours, from about 4 hours to about 36 hours, from about 12 hours to about 24 hours, from about 2 days to about 30 days, from about 5 days to about 20 days, from about 7 days or more, from about 10 days or more, from about 100 days or more; from about 1 week to about 4 weeks, from about 1 month to about 24 months, from about 2 months to about 12 months, from about 3 months to about 9 months, from about 1 month or more, from about 2 months or more, or from about 6 months or more.

Accordingly, in another aspect, an implantable device is contemplated. The device comprises a reservoir comprising a formulation of a small molecule therapeutic agent, the formulation comprising (i) an amount of the therapeutic agent to provide substantially zero-order release of the therapeutic agent for a delivery period of at least about 30 days and at a rate that provides a therapeutic effect and (ii) an organic acid that (a) maintains a pH of the formulation when hydrated in its environment of use of between 3.0-6.0 for the delivery period, (b) is present in a stoichiometric (molar) excess relative to the therapeutic agent, and (c) is present at the end of the delivery period in an amount approximately equal to or above its saturation concentration in the formulation when hydrated.

In another aspect, an implantable device is contemplated. The device consists of a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) an organic acid that (a) maintains a pH of the formulation when hydrated in its environment of use that is approximately equal to or less than the pKa of the protonated drug for the delivery period; (b) is present in stoichiometric (molar) excess, relative to the therapeutic agent, and (c) is present at the end of the delivery period in an amount approximately equal to or above its saturation concentration in the formulation when hydrated.

In one embodiment, the formulation comprising a small molecule therapeutic agent and a stoichiometric excess of an organic acid is in a dry form. For example, the dry formulation may be present in the reservoir of a device as a powder, a tablet or a film. The device when in use, in vitro or in vivo, imbibes fluid from the surrounding environment to hydrate the dry formulation, thus forming in situ an aqueous suspension containing particles of both the salt form of the therapeutic agent and undissolved excess acid.

The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are preferred because of convenience in implantation and removal of the drug delivery device. Exemplary subcutaneous delivery sites include under the skin of the arm, shoulder, neck, back, or leg. Sites within a body cavity are also suitable implantation sites. Methods for implanting or otherwise positioning drug delivery devices for subcutaneous delivery of a drug are well known in the art. In general, placement of the drug delivery device will be accomplished using methods and tools that are well known in the art, and performed under aseptic conditions with at least some local or general anesthesia administered to the subject.

### Methods of Treatment

In other aspects, methods of treatment using the compositions and devices described herein are contemplated. In one embodiment, a method for sustained, controlled delivery of a central nervous system medication is contemplated, where a composition or a delivery device comprising a composition as described herein is provided.

In another embodiment, a method for sustained, controlled delivery of an antipsychotic medication is contemplated, where a composition or a delivery device comprising a composition as described herein is provided.

In another embodiment, a method for maintaining therapeutic plasma levels of an antipsychotic medication is contemplated, thus delaying relapse for stable, previously medicated patients for at least 4 weeks is contemplated.

Based on the foregoing, the compositions described herein comprised of a small molecule therapeutic agent and an organic acid provide release of the therapeutic agent for an extended period of time—for at least about 14 days or for at least about 30 days—at a constant rate that approaches zero-order release kinetics for the period. The composition comprises the therapeutic agent in an amount sufficient for a therapeutic dose of the agent for period, and an amount of the organic acid to maintain either (i) a concentration of the protonated therapeutic agent at or near its saturation concentration in the hydrated composition for the period and/or (ii) a concentration of the organic acid equal to or above its saturation concentration in the hydrated composition at the end of the delivery period. The near-saturated concentration of drug is with respect to the aqueous phase of the composition. The composition is, in some embodiments, retained in a drug delivery system (or device) and when placed in an environment of use (such as a subcutaneous implantation site, e.g., plasma or interstitial fluid with a constant pH ˜7.4) produces a constant concentration gradient between the device interior and its environment of use that facilitates a constant release rate (near zero-order kinetics) of the therapeutic agent over time.

### III. Examples

The following examples are illustrative in nature and are in no way intended to be limiting.

### Example 1

### Formulation Comprising Risperidone as a Small Molecule Therapeutic Agent and an Organic Acid

Risperidone was compounded with p-aminobenzoic acid (PABA) at acid:drug ratios of 1:1, 1.5:1, or 2:1 (molar basis), tableted with lactose binder (13%), and loaded into delivery devices equipped with 0.1 micron polyvinylidene fluoride (DURAPORE®) membranes. In some devices, approximately 50% of the available membrane surface area was blocked to measure the influence of surface area upon output rate. All devices were vacuum back-filled with phosphate buffer and transferred to jars containing a volume (˜100 mL) of the same buffer. The sealed jars were then incubated at 37° C., and small aliquots (˜500 μL) of receiving buffer were withdrawn at selected time points to quantify the released drug by high pressure liquid chromatography (HPLC). Release of risperidone is shown in FIG. 2.

### Example 2

### Formulation Comprising Olanzapine as a Small Molecule Therapeutic Agent and an Organic Acid

Olanzapine was compounded with p-aminobenzoic acid (PABA) or with p-toluic acid at acid:drug ratios of 1.5:1 (molar basis), tableted with lactose binder (13%), and loaded into delivery devices equipped with 0.1 micron polyvinylidene fluoride (DURAPORE®) membranes. Devices were vacuum back-filled with phosphate buffer and transferred to jars containing a volume (˜100 mL) of the same buffer. The sealed jars were then incubated at 37° C., and small aliquots (˜500 μL) of receiving buffer were withdrawn at selected time points to quantify the released drug by high pressure liquid chromatography (HPLC). Release of olanzapine is shown in FIG. 3A.

### Example 3

### In Vivo Pharmacokinetics of 12-Month Implant Devices Loaded with a Formulation Comprising Risperidone and Para-Aminobenzoic Acid

Risperidone base (75.00 g, 0.1827 mol) was weighed and transferred to a 1.0 L media bottle containing a stir bar. PABA (50.00 g, 0.3646 mol) was weighed and added to the bottle containing risperidone. Approximately 750 mL of methanol was then added. The bottle containing the formulation was sealed and mixed via magnetic mixer. The mixture was inspected visually for full dissolution of the drug and acid, and the stir bar was removed. The solution was then filtered (0.45μ DURAPORE®) directly into a rotary evaporator and allowed to undergo a primary drying step under vacuum until the bulk of the solvent was evaporated, with the start and end times recorded. After completion of rotary (primary) drying, the vacuum was released, and the resulting foamy material was briefly reduced by hand before being subjected to a secondary drying under high vacuum.

Following secondary drying, all mixtures were transferred to a glove box for pulverization. Formulations were transferred into a grinding chamber equipped with a blade for grinding dry materials and ground using a 20,000 rpm blender base. To prevent overheating of the formulation a custom-made polypropylene sleeve was used to surround the chamber with dry ice. The mixture was ground for 5 cycles. The resulting powder was mixed with 12% by weight polyvinylperolidone (PVP ˜40K, Sigma Aldrich) as a binding agent and with 1% by weight stearic acid (1% of the final powder mass, Sigma Aldrich) as a lubricant. Tablets were produced using a tablet press and custom die sets obtained from Vanguard Pharmaceutical Machinery (Spring, Tex.). Dies used for tableting had diameters matched to the internal diameters of the device reservoirs (4.30 mm).

Drug delivery devices were manufactured from titanium, measuring 40.0 mm in length, and having an internal reservoir. Cap subassemblies (see FIGS. 1C-1K) included a DURAPORE® porous membrane (0.1 micron, Millipore Corp). An assembled cap was affixed to a device reservoir and weighed with another assembled cap to obtain the weight of an empty device. Each reservoir subassembly (reservoir+cap at one end) was manually loaded with tablets using forceps before being capped with a second cap subassembly and weighed again to obtain a tablet fill weight. The average fill weight of each device was 460 mg (which corresponds to 230 mg of risperidone as a free base).

After weighing, the assembled devices were individually placed into 20 mL lyophilization vials. The vials were loosely capped with igloo-style rubber septa and placed into a lyophilizer equipped with a stoppering tray system. The air space within each device and vial was evacuated to a vacuum pressure of <1 torr for no less than 30 minutes before sealing.

During the manufacturing process, efforts were made to maintain a low bioburden during the compounding, device assembly, and trocar assembly process. A final, terminal sterilization of both the filled devices and their implanter tools was performed using electron beam sterilization with a split dose of 25 kGy.

Immediately before implantation in vivo, each device was back-filled with sterile phosphate-buffered saline (PBS) using a 20 mL syringe equipped with a blunt fill needle. Upon insertion of the needle through the rubber septa, the vacuum within the vial rapidly drew the hydration solution into the vial and device without any application of manual force to the plunger. After hydration, the needle was withdrawn from the septum, and the device was left for approximately 10 minutes. Each device was then retrieved from its vial, wiped with a tissue to absorb any external fluid, and weighed. Animals were implanted subcutaneously in the dorsum to one side of the midline using a custom implanter tool and the incision closed with a suture or surgical glue. Whole blood samples were obtained for pharmacokinetic (PK) analysis and local safety was assessed for six months. The implant was well tolerated by all animals. PK results are shown in FIG. 4 for the first 6 months. Plasma levels of risperidone active moiety (risperidone plus its active metabolite 9-OH risperidone) peaked in the first few days and then reached a steady state plasma level of about 50 ng/mL for the entire 6-month implantation period. Mass balance analysis revealed that devices removed after 6 months released drug at an average rate of 0.70 mg/day and contained an average of 108 mg of unreleased risperidone. These findings indicate that the devices would have operated for another 154 days in vivo for a total operating period of 337 days. To extend the time period of operation the device reservoir can be sized and filled with drug and organic acid sufficient for the period of delivery at a desired rate. For example, to create a 12-month system the reservoir length is increased by 10% from 40.0 mm to 44.0 mm. Accordingly, the dose rate is scaled by increasing the diameter of the device, or by implanting more than one device per subject.

### Example 4

### In Vivo Pharmacokinetics of 7-Month Implant Devices Loaded with a Formulation Comprising Risperidone and Sebacic Acid

Risperidone base (75.00 g, 0.1827 mol) was weighed and transferred to a 1.0 L media bottle containing a stir bar. Sebacic acid (74.91 g, 0.3704 mol) was weighed and added to the bottle containing risperidone. Approximately 75 mL of methanol was then added. The bottle containing the formulation was sealed and mixed via magnetic mixer. The mixture was inspected visually for full dissolution of the drug and acid, and the stir bar was removed. The mixture was dried, granulated, tableted, loaded into device reservoirs and terminally sterilized as described in Example 3. The device reservoir size was 41.4 mm in length with an inner diameter of 3.6 mm and an outer diameter of 5.21 mm. Five devices were filled with an average of 400 mg of tablets (corresponding to 167 mg equivalents of risperidone base).

Each device was then retrieved from its vial, wiped with a tissue to absorb any external fluid, and weighed. Animals were implanted subcutaneously in the dorsum to one side of the midline using a custom implanter tool and the incision closed with a suture or surgical glue. Whole blood samples were obtained for PK analysis and local safety was assessed for six months. The implant was well tolerated by all animals. PK results are shown in FIG. 4.

### Example 5

### In Vitro Release of Risperidone from Devices Loaded with Various Risperidone Addition Salts

Various salts of risperidone were prepared by dissolving the drug and a two-fold molar excess of the selected acid in methanol. The solvent was removed under reduced pressure. The dried cake was further dried, pulverized, tableted (in some cases), filled into reservoirs, capped and vacuum vialed as described in Example 3. The loaded devices were hydrated and placed in 100 mL of PBS at 37° C. on a planetary rotator (50 rpm). Aliquots of the receiving buffer were analyzed for risperidone concentration (spectrophotometer or HPLC). FIG. 5 presents the cumulative in vitro release (expressed as the percent of total loaded drug released into a receiving medium) for the various risperidone salts.

1. A composition, comprising:
an aqueous suspension comprising
a therapeutic agent that (i) has a water solubility at room temperature of less than 1.0 g/L and (ii) is an organic base, and
an organic acid that (i) has a water solubility at room temperature between 0.1 and 10 g/L, (ii) has a molar mass of less than 500 grams per mole, (iii) is present in a stoichiometric (molar) excess relative to the therapeutic agent, and (iv) maintains a pH of the suspension in its environment of use of between 3.0-6.5 for a period of at least about 30 days.

2. The composition of claim 1, wherein a saturated aqueous solution of the organic acid has a pH value approximately equal to or less than the pKa of the protonated therapeutic agent.

3. The composition of claim 1, wherein the organic acid is present in an amount approximately equal to or above its saturation concentration at the end of the period.

4. The composition of claim 1, wherein the organic acid is present in a stoichiometric excess of 105% to 1000% relative to the therapeutic agent.

5-7. (canceled)

8. The composition of claim 1, wherein the therapeutic is risperidone, olanzapine, asenapine, aripiprazole, or brexpiprazole.

9-10. (canceled)

11. The composition of claim 1, wherein the organic acid is an aromatic carboxylic acid.

12-13. (canceled)

14. The composition of claim 11, wherein the carboxylic acid is one having a carboxylic acid group bound to an unsubstituted benzene or pyridine ring.

15. The composition of claim 14, wherein the carboxylic acid is selected from the group consisting of benzoic acid, picolinic acid, nicotinic acid, and isonicotinic acid.

16. The composition of claim 14, wherein the carboxylic acid is one having a benzene ring and one electron-donating group with antioxidant properties.

17. The composition of claim 16, wherein the carboxylic acid is selected from the group consisting of o-anisic acid, m-anisic acid, p-anisic acid; p-aminobenzoic acid (PABA), o-aminobenzoic acid (anthranilic acid), o-toluic acid, m-toluic acid, p-toluic acid and salicylic acid.

18-25. (canceled)

26. The composition of claim 14, wherein the carboxylic acid is one having one or two carboxylic acid groups directly bonded to a biphenyl ring system.

27. The composition of claim 26, wherein the carboxylic acid is selected from the group consisting of 2-phenylbenzoic acid, 3-phenylbenzoic acid, 4-phenylbenzoic acid and diphenic acid.

28. The composition of claim 14, wherein the carboxylic acid is one having one additional electron donating substituents in addition to hydroxyl group on the carboxylic acid moiety.

29. The composition of claim 28, wherein the carboxylic acid is selected from the group consisting of 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

30. The composition of claim 1, wherein the organic acid is one having a carboxylic acid functional group separated from a benzene, pyridine, naphthalene, or quinoline ring by a chain of 1-4 sp3 hybridized carbons.

31. The composition of claim 30, wherein the carboxylic acid is phenylacetic acid or 3-phenylpropionic acid.

32. The composition of claim 1, wherein the organic acid is an aliphatic dicarboxylic acid with 4-8 carbon atoms between the carboxylic acid groups.

33. The composition of claim 32, wherein the carboxylic acid is selected from the group consisting of adipic acid (CH2)4(COOH)2), pimelic acid (HO2C(CH2)5CO2H), suberic acid (HO2C(CH2)6CO2H), azelaic acid (HO2C(CH2)7CO2H), and sebacic acid (HO2C(CH2)8CO2H).

34. The composition of claim 1, wherein the organic acid is an unsaturated or polyunsaturated dicarboxylic acids containing 4-10 carbons.

35. The composition of claim 34, wherein the carboxylic acid is selected from the group consisting offumaric acid, trans,trans-muconic acid, cis,trans-muconic acid, and cis,cis-muconic acid.

36. The composition of claim 1, wherein the organic acid is a cis-cinnamic acid or a trans-cinnamic acid.

37. The composition of claim 36, wherein the carboxylic acid is a trans-cinnamic acid with one or two electron-donating groups selected from hydroxy, methoxy, amino, alkylamino, dialkylamino, or alkyl groups.

38. The composition of claim 37, wherein the trans-cinnamic acid is selected from the group consisting o-coumaric acid, m-coumaric acid, p-coumaric acid, o-methylcinnamic acid, m-methylcinnamic acid, p-methylcinnamic acid; o-methoxycinnamic acid, m-methoxycinnamic acid, and p-methoxycinnamic acid, andferulic acid.

39-43. (canceled)

44. The composition of claim 1, wherein the organic acid is a hydroxamic acid.

45. The composition of claim 44, wherein the hydroxamic acid is an aromatic hydroxamic acid containing one hydroxamic functional group bonded directly to an aromatic ring.

46. The composition of claim 45, wherein the aromatic ring is selected from the group consisting of a benzene ring, a pyridine ring, a naphthalene ring, a quinoline ring, and a biphenyl ring.

47-49. (canceled)

50. The composition of claim 44, wherein the hydroxamic acid is a dihydroxamic acid containing two or more hydroxamic acid functional groups bonded directly to a benzene ring, a pyridine ring, a naphthalene ring, a quinoline ring, or a biphenyl ring system.

51-54. (canceled)

55. The composition of claim 1, wherein the organic acid contains an aromatic ring and a carboxylic acid functional group.

56. The composition of claim 55, wherein the carboxylic acid is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and phthalic acid.

57. The composition of claim 56, wherein the hydroxy-derivative of cinnamic acid is m-coumaric acid or p-coumaric acid.

58-59. (canceled)

60. The composition of claim 56, wherein the amino-derivative of benzoic acid is 2-amino-benzoic acid (anthranilic acid) or 4-aminobenzoic acid (para-aminobenzoic acid; PABA).

61-63. (canceled)

64. A device, comprising: a composition according to claim 1, wherein the device is configured for subcutaneous implantation into a mammal.

65-72. (canceled)

73. A method for sustained, controlled delivery of a small molecule therapeutic agent, comprising:
providing a composition according to claim 1.

74-76. (canceled)
